(19) United States
(12) Patent Application Publication
(10) Pub. No.: US 2009/0175858 A1
(43) Pub. Date: Jul. 9, 2009

(54) Crystalline EGFR - matuzumab complex and matuzumab mimetics obtained thereof

(75) Inventors: Kathryn M. Ferguson, Media, PA (US); Thorsten Knoechel, Darmstadt (DE); Judith Schmiedel, Berlin (DE)

Correspondence Address: GOODWIN PROCTER LLP;PATENT ADMINISTRATOR, 53 STATE STREET, EXCHANGE PLACE, BOSTON, MA, 02109-2881, US

(73) Assignees: Merck Patent GmbH, Darmstadt (DE); The Trustees of the University of Pennsylvania, Philadelphia, PA (US)

(21) Appl. No.: 12/286,861

(22) Filed: Oct. 2, 2008

Related U.S. Application Data
(60) Provisional application No. 60/997,502, filed on Oct. 2, 2007.

(51) Int. Cl. A61K 39/395; A61P 35/00; C07K 19/00; C07K 16/28; G01N 33/536; G06F 19/00

(52) U.S. Cl. 424/133.1; 530/391.7; 530/387.7; 436/536; 702/19

(57) ABSTRACT

The invention relates to a crystal complex formed by the extracellular domain of EGF receptor (EGFR) and the Fab fragment of anti-EGFR antibody matuzumab (EMD 72000). Especially the invention relates to the identification of the epitope regions on said EGFR, which the antibody matuzumab recognizes as antigen an to which it specifically binds. The invention relates furthermore to protein, peptide and antibody structures which mimic the binding of matuzumab to its epitope region on EGFR, and may be used therefore, as EGFR antagonists with similar or improved properties as compared to matuzumab.

### REFERENCE TO RELATED APPLICATIONS

This application claims the benefit of and priority to U.S. Provisional Patent Application No. 60/997,502, filed Oct. 2, 2007, the disclosure of which is incorporated by reference herein.

### FIELD OF THE INVENTION

The invention relates to a crystal complex formed by the extracellular domain of EGF receptor (EGFR) and the Fab fragment of anti-EGFR antibody matuzumab (EMD 72000). Especially the invention relates to the identification of the epitope regions on said EGFR, which the antibody matuzumab recognizes as antigen an to which it specifically binds. The invention relates furthermore to protein, peptide and antibody structures which mimic the binding of matuzumab to its epitope region on EGFR, and may be used therefore, as EGFR antagonists with similar or improved properties as compared to matuzumab.

### BACKGROUND OF THE INVENTION

Disruption of signal transduction pathways through pharmacological targeting of relevant components within these pathways has become an effective therapeutic option to treat various types of cancer. Membrane tyrosine kinase receptors in cancer cells are a particularly attractive target, with the epidermal growth factor (EGF) family of membrane receptors emerging as one of the most promising.

The EGF family consists of the epidermal growth factor receptor (EGFR, ErbB1, HER1). EGFR is a 170 kD membrane-spanning glycoprotein containing (1) an amino-terminal extracellular domain comprised of 621 amino acid residues, which includes the ligand-binding domain; (2) a single 23-amino-acid transmembrane-anchoring region which may contribute to stability; and (3) a 542-amino-acid carboxyl-terminal intracellular domain which possesses tyrosine kinase activity that activates cytoplasmic targets. Examples of ligands that stimulate EGFR include epidermal growth factor (EGF), transforming growth factor-α (TGF-a), heparin-binding growth factor (HBGF), 3-cellulin, and Cripto-1. Binding of specific ligands results in EGFR autophosphorylation, activation of the receptor's cytoplasmic tyrosine kinase domain and initiation of multiple signal transduction pathways that regulate tumor growth and survival.

Alterations in the function of receptors from the EGF family, especially deregulation of EGFR, have been shown to be associated with autonomous cell growth, invasion, angiogenic potential and the development of metastases.

Ligands such as EGF-like growth factors activate EGFR through binding to the extracellular domain, which initiates multiple growth-regulatory signaling pathways. The two most relevant pathways are the MAPK (mitogen-activated protein kinase, also known as extracellular signal-regulated kinase [ERK]) mitogenic pathway and the phosphatidylinositol 3-kinase (PI3K)/Akt (also known as protein kinase B [PKB]) pathway.

The MAPK pathway regulates gene transcription and proliferation through activation of numerous substrate molecules in the cytosol, nucleus and plasma membrane. The PI3K/Akt signaling pathway mediates cell survival in that recruitment of Akt to the plasma membrane results in prosurvival signaling due to increased expression of anti-apoptotic signals, decreased expression of proapoptotic signals and activation of mRNA translation.

Oncogenic transformation due to aberrant EGFR signaling can be a consequence of several different mechanisms, including receptor overexpression; activating mutations; alterations in the dimerization process required to induce conformational changes in EGFR that activate the intracellular tyrosine kinase moiety and receptor autophosphorylation; activation of the autocrine growth factor loop; and deficiencies in specific phosphatases. Variant EGFR forms carrying mutations in the extracellular domain have been identified and the EGFR variant III (EGFRvIII) is associated with several types of cancer.

It should be remarked that receptor protein tyrosine kinases, such as EGFR kinase are able to undergo both homo- and heterodimerization, wherein homodimeric receptor combinations are less mitogenic and transforming (no or weak initiation of signaling) than the corresponding heterodimeric combinations (Yarden and Sliwkowski, 2001, Nature Reviews, Molecular cell Biology, volume 2, 127-137; Tzahar and Yarden, 1998, BBA 1377, M25-M37).

The epidermal growth factor receptor (EGFR) is aberrantly activated in a variety of epithelial cancers and has been the focus of much interest as a therapeutic target in anti-cancer therapy. EGFR is one of a family of four receptor tyrosine kinases (collectively known as the ErbB receptors) that is involved in critical cellular processes such as proliferation, differentiation and apoptosis (Hubbard and Miller, 2007). Misregulation of EGFR, through overexpression or mutation, leads to constitutive activity or impaired receptor downregulation and can cause malignant transformation of the cell (Mendelsohn and Baselga, 2006).

Based upon structural studies over the past five years of the extracellular regions of EGFR a model has been proposed for ligand dependent dimerization and activation of EGFR. Dimerization of the extracellular region of EGFR is entirely receptor mediated with the majority of interactions contributed by domain II of EGFR. The crystal structure of an EGF-EGFR extracellular domain complex, wherein the receptor domain exists in dimeric form, has been provided Ogiso, H. et al., 2002, Cell 110, 775-787. The structure of an EGF-EGFR extracellular domain complex obtained by crystallization at low, non-physiological pH, wherein the receptor exists in monomeric form has also been provided (Ferguson, K. M. et al., 2003, Mol Cell 11, 507-517). The structure of a transforming growth factor alpha (TGF-a)-EGFR extracellular domain complex in dimeric form has also been determined (Garrett, T. P. et al., 2002, Cell 110, 763-773). In the unliganded state the receptor adopts a very different conformation that occludes much of the dimerization interface in an intramolecular interaction or tether with domain IV. Upon ligand binding the extracellular region of EGFR undergoes a dramatic domain rearrangement. Additional ligand induced conformational changes stabilize the precise conformation of domain II required for dimerization. Receptor dimerization brings the intracellular tyrosine kinase domains into close proximity promoting their allosterical activation. This mechanism suggests a number of ways to inhibit EGFR activation through interaction with the extracellular region of the receptor X-ray structures and biochemical analysis of receptor-antibody complexes have indicated several modes of binding that leading to effective inhibition of ErbB receptor signaling.

There are several possible strategies to pharmacologically target EGFR, including monoclonal antibodies (MAbs) to compete with activating EGFR ligands for binding to the extracellular domain; small-molecule inhibitors of the intracellular tyrosine kinase domain of the receptor; EGFR ligand-conjugated toxins to deliver toxins into tumor cells; antisense oligonucleotides to reduce EGFR levels; and inhibitors of downstream effectors of EGFR signaling pathways.

The first strategy used clinically to target aberrant EGFR signaling in malignant cells was the use of MAbs. Anti-EGFR antibodies not only disrupt receptor/ligand interactions, blocking aberrant signaling and thus tumor cell proliferation and growth, but they may also modulate anti-tumor effectors via antibody-dependent cellular cyto-toxicity (ADCC). Natural killer (NK) cells mediate ADCC by recognizing the carboxyl-terminal ends of antibody molecules via the low-affinity receptor for IgG, FcyRIIIA/CD16. NK cells therefore can closely interact with antibody-coated tumor cells and destroy cells via necrosis and apoptosis.

The first murine anti-EGFR MAb developed showed good anti-tumor activity in animal models. However, their clinical use was limited due to the high incidence of human antimurine antibodies in patients, resulting in reduced efficacy. In response to this disadvantage, researchers developed chimeric and humanized forms of anti-EGFR MAbs.

Cetuximab (IMC-C225, Erbitux©) a chimeric anti.EGFR antibody, was the first Mab of this type that successfully completed clinical trials and was launched in 2003 as a treatment for several cancers. There are a number of other anti-EGFR antibodies under active clinical development for the treatment of cancer. One of them is matuzumab.

Matuzumab (EMD-72000) is a humanized IgG₁ MAb that binds with high specificity and affinity to EGFR. It has been shown in animal tumor xenograft models to have potent inhibitory activity against human cancers, including head and neck, gastric, pancreatic and lung cancers. Matuzumab was shown to block EGF binding to EGFR, thereby inhibiting downstream signaling pathways, and it may also act via ADCC through FcR binding on immune cells. Matuzumab was selected for further development as a treatment for cancer. Matuzumab exhibited antitumor activity against surgical specimens of EGFR expressing human lung (LXFA629) and gastric (GXF251) adenocarcinomas and pancreas adenosquamous carcinoma (PAXF546) that were insensitive to chemotherapeutic drugs (bleomycin, cisplatin, vindesine, paclitaxel, ifosfamide) and implanted s.c. in nude mice. Treatment with matuzumab (0.5 or 0.5 mg/mouse i.p. once weekly for 2 weeks starting when tumors reached 70-120 mm³) was well tolerated and effective against all 3 tumor types. Complete remissions were observed in 83% and 87%, respectively, of animals bearing gastric and lung carcinomas treated with the higher dose. Marked reductions in pancreatic tumors were observed, such that a mean tumor volume of 31% compared to controls was obtained (27). The anti-tumor efficacy of matuzumab (40 mg/kg biweekly) in mice bearing orthotopic human L3.6 pl pancreatic tumors was shown to be enhanced by simultaneous treatment with to gemcitabine (100 mg/kg biweekly). Treatment with either agent alone caused a reduction in tumor size and lymph node and liver metastases. These effects were markedly enhanced by combination treatment. Treatment with matuzumab alone or in combination with gemcitabine also significantly decreased microvessel density and proliferative indices. Treatment was most effective when administered early after tumor cell injection (28). Results from in vitro and in vivo studies further suggest that the anti-tumor effects of matuzumab involve ADCC.

The amino acid sequences of the variable regions of matuzumab (EMD 72000 also known under IMC-h425) and its anti-tumor efficacy are described also in U.S. Pat. No. 5,558,864 and EP 0531 472 B1, which are incorporated by reference.

The peptide sequence of the complete light chain of matuzumab is as follows: (VL is underlined, CDRs are bold and double underlined):

(SEQ ID NO.: 1)

| | |
|---|---|
| 001 | DIQMTQSPSSLSASVGDRVTITCSASSSVTYMYWYQQKPGKAPKLL |
| | IYDT |
| 051 | SNLASGVPSRFSGSGSGTDYTFTISSLQPEDIATYYCQQWSSHIFT |
| | FGQG |
| 101 | TKVEIKRTVAAPSVFIFPPSDEQLKSGTASVVCLLNNFYPREAKVQ |
| | WKVD |
| 151 | NALQSGNSQESVTEQDSKDSTYSLSSTLTLSKADYEKHKVYACEVT |
| | HQGL |
| 201 | SSPVTKSFNRGEC |

The peptide sequence of the complete heavy chain of matuzumab is as follows: (VH is underlined, CDRs are bold and double underlined):

(SEQ ID NO.: 2)

| | |
|---|---|
| 001 | QVQLVQSGAEVKKPGASVKVSCKASGYTFTSHWMHWVRQAPGQGLE |
| | WIGE |
| 051 | FNPSNGRTNYNEKFKSKATMTVDTSTNTAYMELSSLRSEDTAVYYC |
| | ASRD |
| 101 | YDYDCRYFDYWGQGTLVTVSSASTKGPSVFPLAPSSKSTSGGTAAL |
| | GCLV |
| 151 | KDYFPEPVTVSWNSGALTSGVHTFPAVLQSSGLYSLSSVVTVPSSS |
| | LGTQ |
| 201 | TYICNVNHKPSNTKVDKKVEPKSCDKTHTCPPCPAPELLGGPSVFL |
| | FPPK |
| 251 | PKDTLMISRTPEVTCVVVDVSHEDPEVKFNWYVDGVEVHNAKTKPR |
| | EEQY |
| 301 | NSTYRVVSVLTVLHQDWLNGKEYKCKVSNKALPAPIEKTISKAKGQ |
| | PREP |
| 351 | QVYTLPPSRDELTKNQVSLTCLVKGFYPSDIAVEWESNGQPENNYK |
| | TTPP |
| 401 | VLDSDGSFFLYSKLTVDKSRWQQGNVFSCSVMHEALHNHYTQKSLS |
| | LSPG |
| 451 | K |

WO 2006/009694 discloses a crystalline complex formed by the soluble extracellular domain of EGFR with the Fab fragment of cetuximab thus identifying the epitope domain on EGFR recognized by cetuximab.

Since it could be shown (see WO 2004/032960) that the combination of matuzumab with cetuximab elicits a synergistic effect in in-vitro models, it can be assumed that these two antibodies bind to different epitopes on EGFR.

So it is of specific interest to identify the epitope of matuzumab in order to identify and develop peptides, proteins and antibody molecules which are mimetics of matuzumab, that can bind to the same epitope of matuzumab and act as antagonists of the natural ligands of EGFR.

Accordingly, the present invention provides methods for identifying potential mimetics of matuzumab by screening against at least a subset of the coordinates obtained from a respective crystal complex. Mimetics to matuzumab may be assayed for biological activities to obtain EGFR antagonists useful for treatment of EGFR dependent conditions or diseases. EGFR antagonists interact with the receptor to inhibit EGFR tyrosine kinase activity, without limitation, by blocking ligand binding, inhibiting receptor dimerization, ultimately inhibiting receptor substrate phosphorylation, gene activation, and cellular proliferation. Preferably, the antagonists have substantially similar or improved effectiveness as compared to matuzumab. The antagonists are used for treatment of conditions associated with EGFR expression. Such diseases include preferably tumors that express, or overexpress EGFR and which may be stimulated by a ligand of EGFR. Also included are hyperproliferative diseases stimulated by a ligand of EGFR.

It is a further goal of the invention to combine such matuzumab mimetics with cetuximab or peptides, proteins or antibody molecules other than cetuximab that bind to the known epitope of cetuximab, which is—according to the results of this invention—different from the epitope of matuzumab. By specifically blocking the epitopes on EGFR recognized by these two antibodies (matuzumab and cetuximab) with these antibodies or specifically designed mimetics of these antibodies having similar or improved binding properties towards these epitopes, an improved therapy of EGFR related diseases and disorders should be achieved, since it cannot be assumed per se, that native antibodies like matuzumab or cetuximab have optimum binding affinities. Thus, the knowledge of important epitopes, which block ligand binding and abolish or reduce tyrosine kinase signaling, is of special importance in order to be able to design and develop novel epitope binders with similar or preferably improved properties as compared to matuzumab and optionally cetuximab.

### SUMMARY OF THE INVENTION

The present invention describes for the first time the crystal structure of the Fab fragment of matuzumab (Fab72000) bound to a truncated form of the extracellular region of EGFR. By means of this structure it was found that matuzumab binds to a novel epitope on domain III of EGFR that is distinct from both the ligand and cetuximab binding regions on that domain. Based on this structure the inventors propose a novel mechanism of inhibition of EGFR activation driven by sterical hindrance of the EGF to adopt the conformation required for dimerization. This novel mechanism has implications for both the application of current drugs and the development of new anti-EGFR therapeutics.

In one aspect, the present invention provides a crystal of a receptor-antibody complex comprising a receptor-antibody complex of an epidermal growth factor receptor (EGFR) extracellular domain and cetuximab Fab, wherein the crystal has a resolution determined by X-ray crystallography of better than about 5.0 Angstroms (Å). Preferably, the crystal has a resolution determined by X-ray crystallography of better than about 4.0 Angstroms, more preferably better than about 3.0 Angstroms.

The crystal belongs to space group C2 and has unit cell dimensions a=141 Å(±3 Å), b=205 Å(±3 Å), c=81 Å(±3 Å), and β=117°(±1°). Preferably the crystal complex has the following unit cell dimensions: a=141.1 Å (±0.3 Å), b=205.0 Å(±0.3 Å, c=81.6 Å (±0.3 Å), and β=117.5° (+0.5°).

In another aspect the invention provides the epitope region on the EGF receptor domain III to which matuzumab, preferably the Fab fragment of matuzumab specifically binds. This epitope includes one or more amino acid residues located at positions in the range between positions 433 to 466 on the EGF receptor, especially and in detail Ser433, Asp434, Ala448, Asn449, Asn452, Trp453, Lys454, Lys455, Leu456, Phe457, Gly458, Thr459, Ser460, Gly461, Lys463, Thr464 and Ile466. It could be found that preferably the complete sequence track between positions Trp452 and Thr464 seems to be recognized as antigen region for matuzumab. This sequence track corresponds to the loop of the domain III beta helix of EGFR. In contrast to that, cetuximab interacts with amino acids outside this sequence track, which are Gln384, Gln408, Ser418, Ser440, Lys465, Ser468 and Asn469 (WO2006/009694).

At least the following amino acids in the CDR regions or CDR near regions of matuzumab are involved in interactions/binding to above-specified receptor amino acid residues:

| | |
|---|---|
| CDR1, light chain: | Tyr31 |
| CDR2, light chain: | Asp49 |
| CDR3, light chain: | Trp90, His93 |
| CDR1, heavy chain: | (Thr30), Ser31, Trp33, His35 |
| CDR2, heavy chain: | Asn52, Ser54, Glu50, Asn55, Asn59 |
| CDR3, heavy chain: | Tyr103, Tyr101, Asp100 |

Position Lys454 of the receptor interacts preferably with the following amino acids of matuzumab: Ser54, Ser31, Asp100, Tyr101, Asn52, Ser54, Asn55 (all heavy chain).

Position Lys455 of the receptor interacts preferably with the following amino acids of matuzumab: Ser54, Asn55 (all heavy chain).

Position Leu456 of the receptor interacts preferably with the following amino acids of matuzumab: Asn55 (heavy chain).

Position Phe457 of the receptor interacts preferably with the following amino acids of matuzumab: Trp33, Asn52, Asn55 (all heavy chain).

Position Gly458 of the receptor interacts preferably with the following amino acids of matuzumab: Trp33, Asn59 (all heavy chain)

Position Thr459 of the receptor interacts preferably with the following amino acids of matuzumab: Trp33, Glu50 (all heavy chain), Trp90, His93 (all light chain).

Position Ser460 of the receptor interacts preferably with the following amino acids of matuzumab: Trp33, His35, Glu50, Tyr101 (all heavy chain).

Position Gly461 of the receptor interacts preferably with the following amino acids of matuzumab: Trp90, Tyr31 (all light chain).

Position Lys463 of the receptor interacts preferably with the following amino acids of matuzumab: Tyr31, Asp49 (all light chain), Tyr101 (heavy chain).

Position Thr464 of the receptor interacts preferably with the following amino acids of matuzumab: Tyr103 (heavy chain).

In another aspect, the present invention provides a method of identifying a peptidic mimetic or analog of matuzumab comprising comparing a three-dimensional structure of said mimetic/analog with a three-dimensional structure determined for the above-specified crystal complex. Preferably, the three dimensional structure of the mimetic is compared with at least a subset of the crystal coordinates of matuzumab.

In a further aspect, the invention provides crystal complexes formed by the soluble extracellular EGFR comprising domain III and the Fab fragment of matuzumab or a matuzumab mimetic, wherein binding-relevant atom-atom distances between receptor atoms and matuzumab/matuzumab-mimetic are less than 4 A°, preferably less than 3 A°. In a preferred embodiment the distances for a matuzumab/EGFR complex are the respective values as specified in Table 2.

In one embodiment, identifying a mimetic/analog is carried out by comparing the three-dimensional structure of the mimetic /analog against the coordinates of at least one, preferably more than one EGFR amino acid bound by or essentially interacting with matuzmab Fab. Such EGFR amino acid(s) is (are) selected from the group consisting of Ser433, Asp434, Ala448, Asn449, Asn452, Trp453, Lys454, Lys455, Leu456, Phe457, Gly458, Thr459, Ser460, Gly461, Lys463, Thr464 and Ile466, preferably of Lys454, Lys455, Leu456, Phe457, Gly458, Thr459, Ser460, Gly461, Lys463 and Thr464.

In yet another embodiment, screening is carried out by comparing a three dimensional structure of a mimetic with the atomic coordinates of a region of EGFR selected from the group consisting of about amino acid residue 448 to about amino acid residue 464, preferably amino acid residue 453 or 454 to about amino acid residue 464. In yet another embodiment further amino acid residues of EGFR are included such as Ser433 up to Asn452.

Of specific interest and quality are mimetics of matuzumab which bind to or interact with the following positions on the EGF receptor, or at least bind to or interact with the following positions on the EGF receptor:
(1) Lys454
(2) Lys454 and Lys455
(3) Lys454 and Lys455 and Phe457
(4) Lys454 and Lys455 and Phe457 and Gly458
(5) Lys454 and Lys455 and Phe457 and Gly458 and Thr459
(6) Lys454 and Lys455 and Phe457 and Gly458 and Thr459 and Ser460
(7) Lys454 and Lys455 and Phe457 and Gly458 and Thr459 and Ser460 and Gly461
(8) Lys454 and Lys455 and Phe457 and Gly458 and Thr459 and Ser460 and Gly461 and Lys463
(9) Lys454 and Lys455 and Phe457 and Gly458 and Thr459 and Ser460 and Gly461 and Lys463 and Thr464
(10) Phe457
(11) Phe457 and Lys454
(12) Phe457 and Lys454 and Gly458
(13) Phe457 and Lys454 and Gly458 and Thr459
(14) Phe457 and Lys454 and Gly458 and Thr459 and Ser460
(15) Phe457 and Lys454 and Gly458 and Thr459 and Ser460 and Gly461
(16) Thr459
(17) Thr459 and Lys454
(18) Thr459 and Lys454 and Phe457
(19) Thr459 and Lys454 and Phe457 and Gly458
(20) Thr459 and Lys454 and Phe457 and Gly458 and Ser460
(21) Thr459 and Lys454 and Phe457 and Gly458 and Ser460 and Gly461
(22) Ser460 and Lys454
(23) Ser460 and Lys454 and Phe457
(24) Ser460 and Lys454 and Phe457 and Thr459
(25) Ser460 and Lys454 and Phe457 and Thr459 and Gly461
(26) Lys454 and Phe457
(27) Lys454 and Phe457 and Ser460
(28) Lys454 and Phe457 and Gly461
(29) Lys454 and Phe457 and Thr459
(30) Lys454 and Phe457 and Thr459 and Ser460
(31) Lys454 and Phe457 and Thr459 and Ser460 and Gly461
(32) Phe457 and Ser460
(33) Phe457 and Thr459 and Ser 460
(34) Phe457 and Thr459 and Ser 460 and Gly461
(35) Ala448
(36) Ala448 and Asn449
(37) Ala448 and Lys454
(38) Ala448 and Lys454 and Phe457
(39) Ala448 and Lys454 and Phe457 and Thr459
(40) Ala448 and Lys454 and Phe457 and Ser460
(41) Ala448 and Lys454 and Phe457 and Gly461
(42) Ala448 and Lys454 and Phe457 and Thr459 and Ser460
(43) Ala448 and Lys454 and Phe457 and Thr459 and Ser460 and Gly461
(44) Asn449
(45) Asn449 and Lys454
(46) Asn449 and Lys454 and Phe457
(47) Asn449 and Lys454 and Phe457 and Thr459
(48) Asn449 and Lys454 and Phe457 and Thr459 and Ser460
(49) Asn449 and Lys454 and Phe457 and Thr459 and Ser460 and Gly461
(50) Asn449 and Lys454 and Phe457 and Ser460
(51) any of these elections (1)-(50) with Ser433 and/or Asp433

The mimetic according to the invention may be peptide, a polypeptide, a protein or an immunoglobulin or a fragment thereof. The mimetic may be in some cases a small non- or partially peptidic molecule. In any case, the mimetic has the principal biological properties of matuzumab with respect to inhibiting and blocking the EGF receptor with respect to the natural ligands of EGFR and abolishing or reducing tyrosine kinase activity.

In another aspect of the invention, a mimetic that is an antibody or a fragment thereof is identified by introducing one or more substitutions in at least a single CDR region of matuzumab and/or at non-CDR amino acids of the antibody that interact with the CDR and affect its conformation. In one embodiment, at most a single substitution is made in each CDR, that means CDR1, CDR2 and CDR3, or only in one of the CDRs. In another embodiment, substitution are made solely in CDR3 or at amino acids that affect the conformation of CDR3. In a further preferred embodiment, substitution is carried out in CDR2 and CDR3 and optionally in the environment of the respective CDRs affecting the antibody conformation. Preferably substitutions are made in the heavy chain CDRs.

Preferred positions in the CDRs of matuzumab to be modified in order to obtain improved mimetics are Trp90 and His93 (each CDR3 light chain), Asp100, Tyr101 and Tyr103 (each CDR3 heavy chain), Glu50, Asn52, Ser54 and Asn55 (each CDR2 heavy chain), Ser31 and Trp33 (each CDR1 heavy chain).

In a further aspect of the present invention provides identifying mimetics of matuzumab which can be therapeutically combined with cetuximab or mimetics of cetuximab as provided by WO 2006/009694, which is incorporated herewith by reference. In one embodiment a pharmaceutical combination is provided of matuzumab mimetics identified in this invention and characterized by its binding /interaction activities with the EGFR epitope residues as depicted in the listing (1)-(51) and cetuximab mimetics characterized by its binding/interaction activities with one or more of the EGFR epitope residues Gln384, Gln408, Ser418, Ser440, Lys465, Ser468 and Asn469. Therefore, another aspect of the invention is a composition of compounds comprising a matuzumab mimetic compound, preferably an antibody and a second compound that

(i) inhibits tyrosine kinase activity of the EGF receptor,
(ii) inhibits dimerization of EGF receptor,
(iii) blocks binding of EGF to EGF receptor, and
(iv) binds or interacts with the following amino acid residues of EGFR or a subset of at least four amino acids:
Gln 384, Gln 408, Ser 418, Ser 440, Lys 465, Ser 468, and Asn 469.

The invention further provides a method for synthesizing the mimetic as described and assaying its binding or physiological activity to select EGFR antagonists useful for inhibiting EGFR function and treating EGFR-associated diseases or conditions. In an aspect of the invention, a mimetic is provided that inhibits tyrosine kinase activity of the receptor. In another aspect of the invention, the mimetic inhibits dimerization of EGFR expressed by a cell. Preferably, the mimetic blocks binding of EGF to EGFR. Mimetics of the invention bind to EGFR and inhibit EGFR functional activity, preferably to a similar or greater extent than matuzumab and optionally cetuximab in an combination approach.

In detail, the invention further provides a method of identifying and selecting a mimetic candidate of matuzumab, preferably a peptide, polypeptide, protein or immunoglobuline, more preferably an targeting antibody or antibody fragment, having similar or the same biological activity as matuzumab; said method comprising comparing (a) the binding coordinates of the mimetic compound or a fragment thereof bound to EGFR within the crystalline structure of a mimetic—EGFR complex with (b) the binding coordinates of the Fab fragment of matuzumab bound to EGFR within the crystalline structure of the matuzumab—EGFR complex as specified in claim 1 or claim 4, wherein said binding coordinates are defined by

(i) positions of amino acid residues of EGFR binding to or interacting with the Fab fragment of matuzumab; the positions are selected from the following amino acids or groups of at least three, four, five or six amino acid residues:
Ser433, Asp434, Ala448, Asn449, Asn452, Trp453, Lys454, Lys455, Leu456, Phe457, Gly458, Thr459, Ser460, Gly461, Lys463, Thr464 and Ile466, or more specific:
(a1) Lys454
(a2) Lys454 and Lys455
(a3) Lys454 and Lys455 and Phe457
(a4) Lys454 and Lys455 and Phe457 and Gly458
(a5) Lys454 and Lys455 and Phe457 and Gly458 and Thr459
(a6) Lys454 and Lys455 and Phe457 and Gly458 and Thr459 and Ser460
(a7) Lys454 and Lys455 and Phe457 and Gly458 and Thr459 and Ser460 and Gly461
(a8) Lys454 and Lys455 and Phe457 and Gly458 and Thr459 and Ser460 and Gly461 and Lys463
(a9) Lys454 and Lys455 and Phe457 and Gly458 and Thr459 and Ser460 and Gly461 and Lys463 and Thr464
(a10) Phe457
(a11) Phe457 and Lys454
(a12) Phe457 and Lys454 and Gly458
(a13) Phe457 and Lys454 and Gly458 and Thr459
(a14) Phe457 and Lys454 and Gly458 and Thr459 and Ser460
(a15) Phe457 and Lys454 and Gly458 and Thr459 and Ser460 and Gly461
(a16) Thr459
(a17) Thr459 and Lys454
(a18) Thr459 and Lys454 and Phe457
(a19) Thr459 and Lys454 and Phe457 and Gly458
(a20) Thr459 and Lys454 and Phe457 and Gly458 and Ser460
(a21) Thr459 and Lys454 and Phe457 and Gly458 and Ser460 and Gly461
(a22) Ser460 and Lys454
(a23) Ser460 and Lys454 and Phe457
(a24) Ser460 and Lys454 and Phe457 and Thr459
(a25) Ser460 and Lys454 and Phe457 and Thr459 and Gly461
(a26) Lys454 and Phe457
(a27) Lys454 and Phe457 and Ser460
(a28) Lys454 and Phe457 and Gly461
(a29) Lys454 and Phe457 and Thr459
(a30) Lys454 and Phe457 and Thr459 and Ser460
(a31) Lys454 and Phe457 and Thr459 and Ser460 and Gly461
(a32) Phe457 and Ser460
(a33) Phe457 and Thr459 and Ser 460
(a34) Phe457 and Thr459 and Ser 460 and Gly461
(a35) Ala448
(a36) Ala448 and Asn449
(a37) Ala448 and Lys454
(a38) Ala448 and Lys454 and Phe457
(a39) Ala448 and Lys454 and Phe457 and Thr459
(a40) Ala448 and Lys454 and Phe457 and Ser460
(a41) Ala448 and Lys454 and Phe457 and Gay461
(a42) Ala448 and Lys454 and Phe457 and Thr459 and Ser460
(a43) Ala448 and Lys454 and Phe457 and Thr459 and Ser460 and Gly461
(a44) Asn449
(a45) Asn449 and Lys454
(a46) Asn449 and Lys454 and Phe457
(a47) Asn449 and Lys454 and Phe457 and Thr459
(a48) Asn449 and Lys454 and Phe457 and Thr459 and Ser460
(a49) Asn449 and Lys454 and Phe457 and Thr459 and Ser460 and Gly461
(a50) Asn449 and Lys454 and Phe457 and Ser460
(a51) any of the elections (a1)-(a50) with Ser433 and/or Asp433
   (ii) the distances of specific atoms within the EGFR amino acids of these positions to specific atoms within the mimetic compound that bind or interact with said amino acid residues of EGFR of these positions, and
   (iii) for reference, the distances of specific atoms within said EGFR amino acids of these positions to specific atoms within the matuzumab Fab fragment, wherein said distances between atoms of the amino EGFR acid residues of said positions and atoms of the mimetic compound binding or interacting with them in said crystal complex are less than 4 Å, preferably less than 3 Å and in some cases less than 2 Å.

In another aspect, the present invention provides a compound, which is not matuzumab (EMD 72000), or a matuzumab mimetic compound, that compound

(i) inhibits tyrosine kinase activity of the EGF receptor,
(ii) inhibits dimerization of EGF receptor,
(iii) blocks binding of EGF to EGF receptor, and
(iv) binds or interacts with at least three amino acid residues of the sequence track between position 448 and 464 of EGFR, preferably between position 454 and 459 of EGFR. More preferably the compound binds or interacts with all of the amino acids of EGFR:

### Lys454, Lys455, Leu456, Phe457, Gly458, Thr459, Ser460, Gly461, Lys463 and Thr464,

especially with:

### Lys454, Lys455, Leu456, Phe457, Gly458 and Thr459.

In a further aspect of the invention a method is provided of designing an anti-EGFR antibody that—

inhibits tyrosine kinase activity of the EGF receptor,

inhibits dimerization of EGF receptor,

blocks binding of EGF to EGF receptor, and

derives from matuzumab (EMD 72000),

whereby the method comprises
(i) substituting at least a single amino acid in the CDR regions of the heavy and/or light chain of matuzumab or in the framework regions (FR) adjacent to the CDRs with another amino acid,
(ii) preparing a crystalline complex formed by the Fab fragment of said modified matuzumab and the soluble extracellular domain III of EGFR (sEGFRd3),
(iii) comparing the atomic coordinates of the so-formed crystalline complex with respect to specific amino acid residue positions within the EGFR domain as specified above (for example sequences a1-a50), with the corresponding coordinates of a reference crystal complex formed by the non-modified matuzumab with sEGFRd3,
(iv) selecting the modified matuzumab with atomic coordinates that let assume a closer interaction with respect to said specific amino acid residue positions in the EGFR domain,
(v) assaying its binding affinity and biological activity by means of standard methods, and
(vi) optionally repeating steps (i) to (v).

In another aspect, the present invention provides a matuzumab mimetic identified by the methods described in this application.

In another aspect, the present invention provides pharmaceutical compositions and methods of inhibiting EGFR comprising administering the identified mimetic molecules.

In another aspect, the present invention provides pharmaceutical compositions and methods of treating a disease or condition associated with EGFR expression comprising administering the identified mimetic.

In one non-limiting embodiment, the present invention provides a method of inhibiting growth of a tumor cell that expresses EGFR comprising administering the identified mimetics.

In another embodiment, the present invention provides pharmaceutical compositions and methods of treating a hyperproliferative diseases stimulated by a ligand of EGFR by means of a matuzumab mimetic.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Matuzumab binds to domain III of EGFR

A. The top panel shows Surface Plasmon Resonance (SPR) analysis of sEGFR and sEGFRd3 binding to immobilized Fab72000. The Fab fragment was covalently coupled to a Biosensor surface over which was passed a series of samples at the indicated concentrations. Representative data sets for sEGFR (black symbols) and sEGFRd3 (red symbols) are shown. The equilibrium SPR response value at each concentration is expressed as a fraction of maximal binding. The curves represent the fit of these data to a simple one-site Langmuir binding equation shown. K_{D} values, based on at least three independent binding experiments, are 103.2±4.0 nM for sEGFR and 50.1±1.3 nM for sEGFRd3.

The bottom panel shows the ability of Fab72000 to compete for binding of sEGFR to immobilized EGF. The equilibrium SPR response obtained upon addition of the indicated excesses of Fab to a 600 nM sample of sEGFR is shown, normalized to the response obtained with no added Fab. For comparison the binding of sEGFR without added Fab and the previously reported value for addition of a 2 fold molar excess of the Fab fragment from cetuximab (FabC225) is shown. (Li et al., 2005). Each data point is the mean of at least three independent measurements.

B. On the left side a cartoon representation of the Fab72000:sEGFRd3 complex is shown. For reference a cartoon of tethered sEGFR with domain III in the same orientation is shown to the right. Domain I is colored in red, domain II in green, and domains III and IV are in gray with the secondary structure elements highlighted in red and green respectively. Fab72000 is colored cyan for the V_{L} chain and yellow for the V_{H}. The box indicates the region of the Fab72000:sEGFRd3 complex shown in C. The arrow indicates the rotations performed for the detailed representation in C.
C. A closeup view of the Fab72000 binding site on domain III of sEGFR. The V_{L} and V_{H} chain are represented in grey with highlights in cyan and yellow respectively. The interacting CDRs are shown in cyan for L2 and L3 of the V_{L} chain, and in yellow for H2 and H3. The side chains of the amino acids participating in key interactions are shown. Amino acids are labeled with a yellow background for those from Fab7200 V_{H}, a cyan background for V_{L} and in black for sEGFRd3. Hydrogen bonds are shown with dashed green lines.

FIG. 2. The matuzumab binding site on domain III does not occlude the EGF/TGFα binding site.

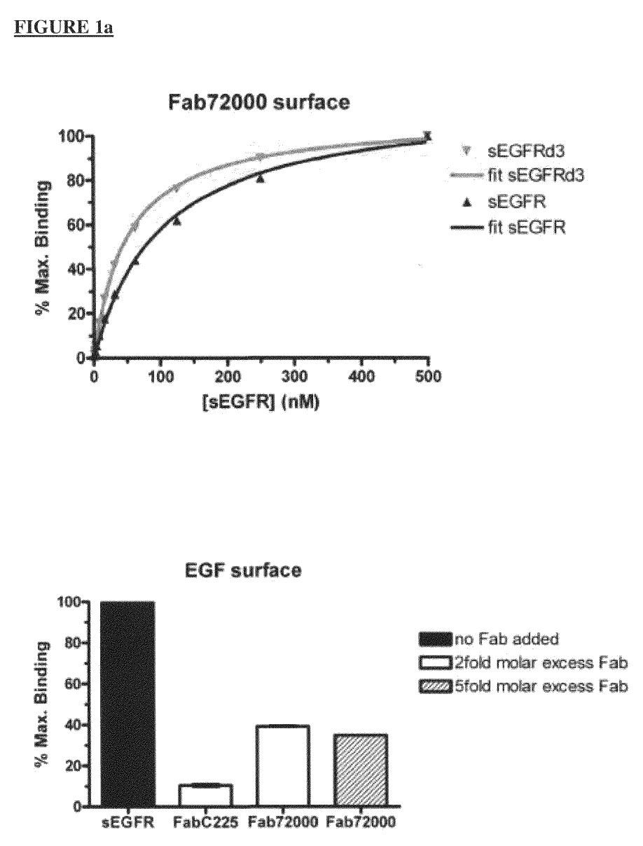

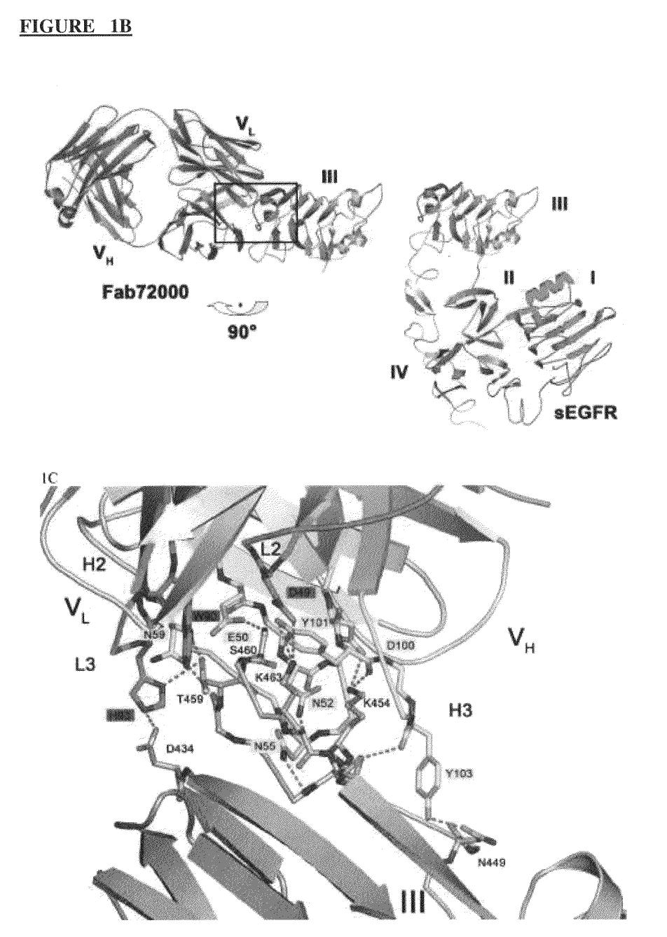

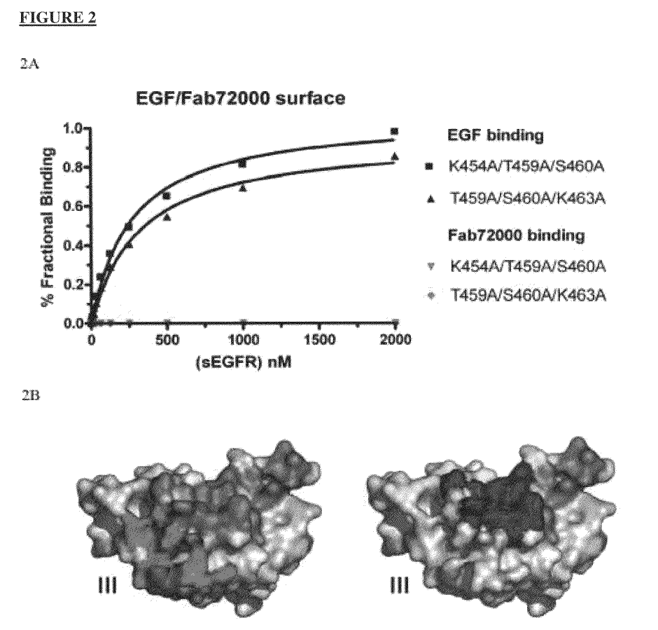

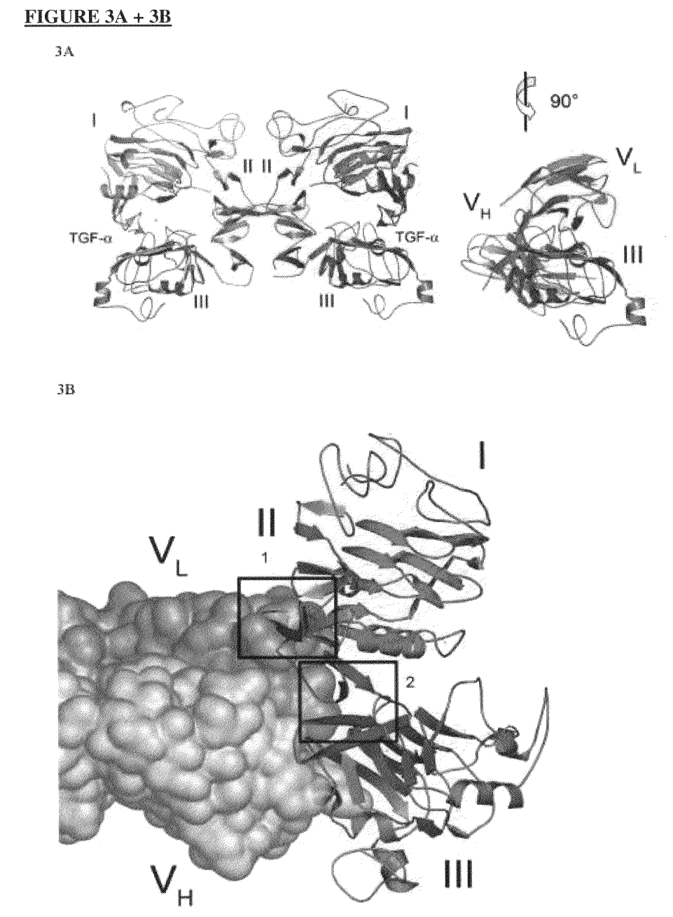

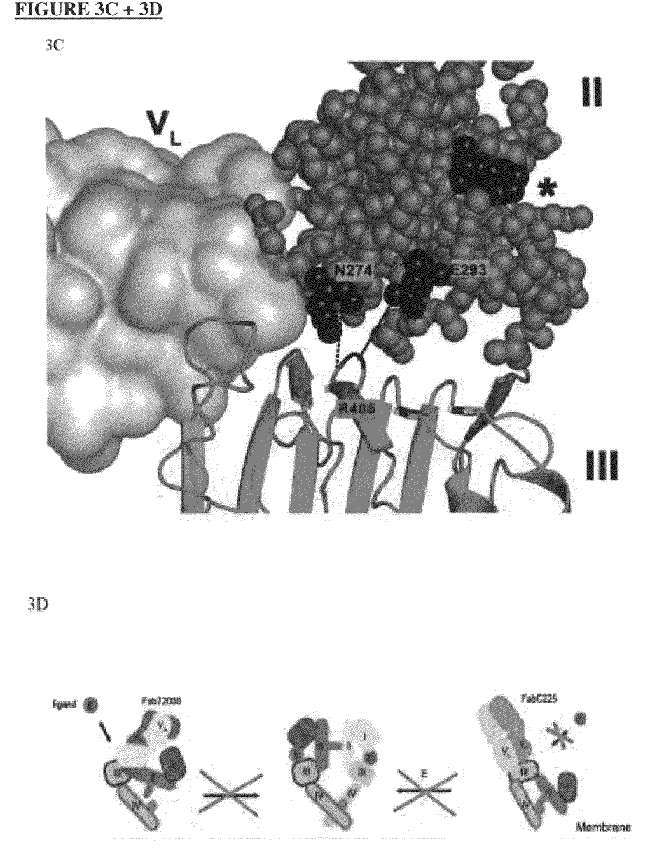

A. Mutational analysis of the matuzumab epitope on sEGFR. Binding of mutated sEGFR to immobilized EGF is shown in black and to a Fab72000 surface in red. Data are presented as described in FIG. 1A. The SPR response is expressed as percent fractional binding of the response for wild-type sEGFR.
B. Surface representation of domain-III in the same orientation as FIG. 1B. The surface is colored indicating on the left side the binding sites of matuzumab (red) and EGF (green). The residues mutated for the binding studies in A. are labeled. On the right side the same orientation of domain III shows the binding sites of cetuximab (yellow), EGF (green) and their overlap (blue).

FIG. 3. Implications for the mechanism of inhibition of EGFR by matuzumab

A. Cartoon representation of the complex Fab72000/sEGFRd3 on the right side and of the EGF bound sEGFR dimer on the left side. Domain III in the Fab7200/sEGFRd3 complex is in the same orientation as the domain III in the right hand protomer of the dimer. Domain III is shown in red/gray, the Fab light chain in cyan and the heavy chain in yellow. In the dimer the ligand is colored in violet, domain I and III in red and domain II in green. The secondary structure elements in domain III are shaded with gray. The arrow shows the rotation to the orientation in part B.
B. Overview of Fab72000 modeled to the extended receptor conformation. The Fab is shown in a surface representation with the light chain colored in cyan and the heavy chain in yellow. The receptor domains are shown as cartoon, domain I colored in red, domain II in green and domain III in red/gray. The N-Terminus of domain I clashes with the light chain as indicated by box 1. Box 2 marks the areas shown in more detail in C.
C. Close-up view of Fab72000 in close proximity to dimer stabilizing domain II/III interactions in the dimerization competent receptor conformation. sEGFR domain II is shown in sphere representation colored in green and domain III as cartoon in red/gray. The crucial amino acids participating in the dimer stabilizing in domainil and III are colored in black. The domain-II loop (residues D279 and H280) exposed into the dimer interface through the domain-II/III interactions is indicated by an asterisk. Fab72000 V_{L} is shown in surface representation in gray. Bonds are shown with dashed black lines. Domain I, IV and V_{H} are not drawn to give a clear view on the interactions.
D. Distinct mechanisms of inhibition of EGFR dimerization by matuzumab and cetuximab.

In the center a cartoon is shown for the ligand induced EGFR dimer, with domain I in red, domain II in green, domain III in gray with red border, domain IV in gray with green border and the ligand (E) in violet, the colors for one protomer are lightened for contrast. Fab72000 binds to domain III of sEGFR and sterically prevents the receptor from adopting the conformation required for dimerization. Importantly Fab72000 blocks the local conformational changes in domain II that are critical for formation of the high affinity productive dimer. The inhibition is non-competitive; the ligand binding site on domain III is not blocked. By contrast FabC225 (cetuximab) is a competitive inhibitor that blocks the ligand binding site on domain III and thus prevents ligand induced receptor dimerization and activation.

### DETAILED DESCRIPTION

The present invention provides a co-crystal of EGFR extracellular domain, especially domain III. The crystal preferably has the space group C2 and the following unit cell dimensions:

a=141.1 Å(±0.3 Å), b=205.0 Å(±0.3 Å), c=81.6 Å(±0.3 Å), and β=117.5°(±0.5°).
Crystallization of the EGFR-matuzumab Fab complex may be carried out from a solution of matuzumab Fab and EGFR with various techniques, such as microbatch, hanging drop, sitting drop, sandwich drop, seeding and dialysis. The solution is prepared by combining EGFR extracellular domain with matuzumab Fab in a suitable buffer. A standard buffering agent such as Hepes, Tris, MES and acetate may be used. The buffer system may also be manipulated by addition of a salt such as sodium chloride, ammonium sulfate, sodium/potassium phosphate, ammonium acetate among others. Imidazole may also be used as a buffer. The concentration of the salt is preferably about 10 mM to about 500 mM, more preferably about 25 mM to about 100 mM, and most preferably about 50 mM. The pH of the buffer is preferably about 6 to about 8, more preferably about 6 to about 7. The concentration of the protein in the solution is preferably that of super-saturation to allow precipitation. The solution may optionally contain a protein stabilizing agent.

In one embodiment, the crystal is precipitated by contacting the solution with a reservoir that reduces the solubility of the proteins due to presence of precipitants, i.e., reagents that induce precipitation. Such contacting may be carried out through vapor diffusion. Examples of precipitants include ammonium sulfate, ethanol, 3-ethyl-2,4 pentanediol, and glycols, particularly polyethanol glycol (PEG). The PEG utilized preferably has a molecular weight of about 400 to about 20,000, more preferably about 3000 Da, with a concentration of about 10% to about 20% , more preferably about 15% (w/v). Some precipitants may act by making the buffer pH unfavorable for protein solubility.

The temperature during crystallization is preferably of about 0° C. to about 30° C., more preferably about 20° C. to about 30° C., and most preferably about 25° C. In addition to generation of structure, the crystallization technique of the invention may also be used to increase purity of proteins.

In some embodiments, the locations of at least some atoms of mimetic compounds of matuzumab that contact EGFR correspond to the locations of atoms of matuzumab that contact EGFR. The correspondence is preferably within the distance range of 1 to 4 Å preferably 1 to 3 Å. The atoms usually interact with EGFR in a manner similar to the corresponding atoms of matuzumab Fab (i.e., polar, basic, acidic, hydrophobic). The mimetics may contain various numbers of such corresponding atoms, and binding of the mimetic to EGFR may be completely or only partially dependent on such corresponding interactions. In certain embodiments, such atomic interactions with EGFR may be supplemented by interactions of other atoms of the mimetic that also interact with EGFR. The binding ability of the mimetics can be evaluated by various Computer methods and programs known in the art.

The mimetics according to the invention bind to EGFR and mimic effects of matuzumab both in vivo and in vitro, preferably its efficacy as EGFR antagonist. The mimetic compounds may be designed based on criteria such as affinity for EGFR, desirable efficacy and/or desirable selectivity. These mimetics have at least a single biological or binding activity of matuzumab. Methods and assays for determining biological and/or binding activity are well known in the art.

The term “mimetic” or “mimetic compound” as used herein includes a compound that mimics the specificity to EGFR. Especially, a mimetic compound inhibits tyrosine kinase activity by blocking EGFR comparable to matuzumab. Preferably a mimetic compound according to the invention is a peptide or a polypeptide or, more preferably, an antibody or a targeting CDR containing fragment thereof. A “mimetic” according to the closest meaning is an antibody different from matuzumab (for example a modified matuzumab) but having similar or identical biological activity in context with EGFR as compared to matuzumab.

After screening and selection of the suitable mimetic compound, the selected mimetic may be synthesized, and various assays carried out to measure the biological or physiological activity of the mimetic select an EGFR antagonist. A preferred EGFR antagonist has one or more of the following properties: inhibits EGFR tyrosine kinase activity; blocks ligand binding to EGFR; inhibits EGFR dimerization (homodimerization with EGFR or heterodimerization with another EGFR family receptor subunit); inhibits EGFR Substrate phosphorylation; inhibits EGFR mediated gene activation; inhibits growth or proliferation of a cell the expresses EGFR. Preferably, the antagonist has substantially similar or improved effectiveness as an EGFR antagonist as compared to matuzumab.

Tyrosine kinase inhibition can be determined using well-known methods; for example, by measuring the auto-phosphorylation level of recombinant kinase receptor, and/or phosphorylation of natural or synthetic Substrates. Thus, phosphorylation assays are useful in determining EGFR antagonists of the present invention. Phosphorylation can be detected, for example, using an antibody specific for phosphotyrosine in an ELISA assay or on a western blot. Some assays for tyrosine kinase activity are described in Panek et al., J. Pharmacol. Exp. Thera. (1997) 283: 1433-44 and Batley et al, Life Sci. (1998) 62: 143-50.

The ability of a mimetic to block ligand binding can be measured, for example, by in vitro competitive assays. Herein, a ligand of EGFR such as EGF is immobilized, and a binding assay is carried to determine the effectiveness of the mimetic to competitively inhibit binding of EGFR to the immobilized ligand.

In vivo assays can also be utilized to determine EGFR antagonists. For example, receptor tyrosine kinase inhibition can be observed by mitogenic assays using cell lines stimulated with receptor ligand in the presence and absence of inhibitor.

In another aspect, the present invention provides methods of treating EGFR-dependent diseases, disorders and conditions in mammals, preferably in humans by administering a therapeutically effective amount of a mimetic of matuzumab.

Matuzumab mimetics of the present invention are especially useful for treating tumors that express or overexpress EGFR. EGFR (over)expressing tumors are characteristically sensitive to EGF present in their environment, and can further be stimulated by tumor produced EGF or TGF-a.

Examples of tumor that express EGFR and are stimulated by a ligand of EGFR include carcinomas, gliomas, sarcomas, adenocarcinomas, adenosarcomas, and adenomas. Such tumors can occur in virtually all parts of the body, including, for example, breast, heart, lung, small intestine, colon, spleen, kidney, bladder, head and neck, ovary, prostate, brain, pancreas, skin, bone, bone marrow, blood, thymus, uterus, testicles, cervix or liver.

In another embodiment of the invention, matuzumab mimetics, preferably matuzumab peptitic or antibody mimetics, can be administered in combination with one or more other anti-neoplastic or cytotoxic agents, such as chemotherapeutic agents, optionally including-radiation. Such treatments are well known in the art. Suitable anti-neoplastic agents can be, for example, alkylating agents or anti-metabolite agents. Examples of alkylating agents include, for example, cisplatin, cyclophosphamide, melphalan, and dacarbazine. Examples of anti-metabolites include, for example, doxorabicin, daunorabicin, paclitaxel, and irinotecan (CPT-11). Further cytotoxic agents are suitable cytokines, such as IL-2. The chemotherapeutic or cytotoxic agents can also be conjugated to the matuzumab mimetic compound or in case, where the cytotoxic agent is a peptide or polypeptide, can be fused to the antibody or peptidic mimetic forming a fusion protein, preferably an immunocytokine.

### Matuzumab Binds to Domain III of EGFR.

To determine the mode of binding of matuzumab to EGFR and to elucidate the mechanism of inhibition of EGFR by this therapeutic antibody, the X-ray crystal structure of the complex between the Fab fragment of this antibody and the extracellular region of EGFR was determined. First, using surface plasmon resonance (SPR/Biacore), the binding of sEGFR to Fab72000 was studied that had been immobilized on a CM5 biosensor chip (see Examples). It was previously shown that the epitope for cetuximab lies exclusively on domain III of sEGFR (Li et al, 2005).

To address whether this is also true for matuzumab the bind of isolated domain III (sEGFRd3; amino acids 310-514 of mature EGFR) to this same immobilized Fab72000 was analyzed. As shown in FIG. 1A, both sEGFR and sEGFRd3 bind to immobilized Fab72000 with a K_{D}-values of 103.2±4.0 nM and 50.1±1.3 nM respectively. Matuzumab, like cetuximab, binds more tightly to sEGFRd3, possibly due to the absence of steric hindrance from the other domains of sEGFR. Using SPR/Biacore we next tested the ability of matuzumab to compete with binding of sEGFR to immobilized EGF (FIG. 1A). In the presence of 2 fold molar excess of Fab72000 the equilibrium SPR response for a 600 nM sample of sEGFR passed over an EGF Biacore surface is reduced by 45% relative to that with no added Fab. Further addition of up to 50 fold excess does not further reduce this binding. This is in contrast to our previous observations for the Fab fragment from cetuximab (Li et al 2005), where addition of 2 fold excess of Fab reduces the sEGFR binding to base line.

To gain further insight into the binding of matuzumab to sEGFR the structures of Fab72000 alone and in complex with the sEGFRd3 (see Experimental Procedures and Table 1) were crystallized.

Fab 72000/sEGFRd3 Binding Site

The Fab72000 binding site on domain III is centered on the loop that precedes the most C-terminal strand of the domain III beta helix (amino acids 454-464; FIG. 1B). This loop penetrates into the cleft between the V_{L} and V_{H} chains of the Fab. The tip of this loop forms a type I beta turn with T459 and S460 from this turn protruding the farthest into the cleft. This mode of binding is unusual for the recognition of a large protein antigen where it is more common for the epitope to comprise of a large flat surface on the antigen, as was observed for the binding of cetuximab to EGFR.

All of the key interactions made by the Fab are from the complementarity determining regions (CDRs). Each of the CDRs contributes to binding to domain III with the majority of the specificity determining contacts coming from CDRs H2, H3 and L3. It is relatively unusual for all CDRs to contribute to antigen binding (Sundberg and Mariuzza, 2002).

The buried loop for sEGFR makes interactions with both the heavy and light chains of the CDR (FIG. 1C). In the center of the epitope the side chain of T459 interacts with the side chains of W90 and H93 from the Fab light chain. Direct interactions from this region to the heavy chain are made from the backbone carbonyl of T459 and the side chain of S460 that interact with the side chains of N59 and E50 from CDR H2 respectively. Lysine residues on either end of this sEGFRd3loop form salt bridge interactions with aspartic acids on the Fab; K454 with D100 on the heavy chain and K463 with D49 on the light chain. Additional contacts to the main chain of this loop of sEGFRd3 from both CDR H2 and H3 complete the extensive network of hydrogen bonds at the center of the Fab7200/sEGFRd3 interface.

Two important interactions are made to regions beyond the loop between amino acids 454-463. A histidine from CDR L3 (H93) interact with D434 of sEGFRd3, while on the other side of the binding site Y103 from the apex of CDR H3 extends down to interact with N449.

A total of 2 salt bridges and 15 hydrogen bonds are involved in the interaction between Fab72000 and sEGFRd3 in an interface that buries 758 Å² of solvent accessible surface on domain III (a total of 1516 Å² of surface is occluded from solvent in the complex). The majority of this buried surface is apolar (74.1%), indicating a high binding free energy due to the exclusion of solvent from the binding interface (Sundberg et al., 2000).

The shape complementarity (sc) parameter for the interface of the Fab72000/sEGFRd3 complex is 0.61. This is lower than typically observed for antigen-antibody interfaces (0.64 to 0.68) (Lawrence and Colman, 1993) and lower than the sc values for pertuzumab, cetuximab and trastuzumab that range from 0.70 to 0.75 (Franklin et al., 2004; Li et al., 2005; Cho et al., 2003). To add here: comment about binding energy or affinity.

The conformation of the Fab72000 is not significantly altered upon receptor binding. The rmsd for Cα positions is 0.56 Å for V_{L} and 0.96 Å for V_{H}. Also the elbow angle is only 4° increased in the bound conformation of Fab72000, which is in the range of dynamic elbow flexibility (Stanfield et al., 2006).

### Mutations in the Receptor Binding Site Abolish Fab72000 Binding but do not Affect EGF Binding

To confirm the epitope of matuzumab as revealed by the crystal structure in solution receptor mutants of key residues in the antibody-receptor interface were generated. These were tested in SPR/Biacore assays for their ability to bind to a Fab72000 chip as well as EGF (FIG. 2A). Triple alanine mutants in the receptor binding site completely abolish the binding of sEGFR to Fab72000, while the binding to EGF is only slightly affected. This is in contrast to the observations for cetuximab, where mutations of the antibody binding site also reduced significantly the binding to the ligand (Li et al., 2005).

Comparison of the sEGFR Binding Sites for Matuzumab, Cetuximab and EGF/TGFα.

In FIG. 2B the epitopes of matuzumab, EGF and cetuximab on domain III of sEGFR are shown. The epitope of matuzumab does not overlap with that of EGF. Indeed, with domain III from the Fab72000/sEGFRd3 complex overlaid on domain III from the sEGFR:EGF complex (PDB id 1IVO) Fab72000 and EGF do not overlap; the closest approach of the Fab and EGF is 9 Å. This is in stark contrast to the situation for cetuximab binding. There is a high degree of overlap between the cetuximab and EGF binding sites on domain III. Steric inhibition of ligand binding is the primary mechanism of cetuximab mediated inhibition of ligand induced dimerization and activation of EGFR (Li et al., 2005). Clearly the mechanism of inhibition of EGFR activation by matuzumab must be different.

### Implications for the Mechanism of Inhibition of EGFR Activation by Matuzumab

To understand how matuzumab can inhibit ligand induced dimerization and activation of EGFR we consider the effect of the binding of Fab72000 upon the formation of the ligand induced dimeric form of the receptor. When domain III from the Fab72000/sEGFRd3 complex is overlaid on domain III from the receptor in its extended, dimerization competent conformation (PDB id 1 MOX) there are clashes between the Fab and both domain I and domain II of the extended receptor (Ogiso et al., 2002; Garrett et al., 2002) (FIG. 3A). With matuzumab bound to domain III of EGFR the receptor would be unable to undergo the domain rearrangement that is required for dimerization. Further, as detailed below, the binding of Fab72000 also blocks the local conformational changes in domain II that are critical for formation of the productive dimer.

The N-terminal region of the domain I in the extended receptor conformation clashes with the light chain of Fab72000 preventing domain I from reaching the position that is required for high affinity ligand binding (FIG. 3B). This is reminiscent in nature and extent to clashes between cetuximab and domain I that were previously implicated as part of the mechanism of inhibition of EGFR dimerization by that antibody (Li et al, 2005). In that case the different orientation of the Fab on domain III positions the heavy chain such as to occlude the N-terminal portion of domain I from its required position in the receptor dimer.

The clashes between domain II of the extended receptor and Fab72000 are novel and significant. With Fab72000 bound to domain III of EGFR it would not be possible for the C-terminal portion of domain II to adopt the conformation observed in the ligand bound dimeric forms of the receptor. As shown in FIG. 3C, if the Fab72000 is docked onto its domain III binding site on an sEGFR molecule in the extended conformation, there are clashes, predominantly with the V_{L} chain of the Fab, along the C-terminal half of domain II. This C-terminal half of domain II forms the binding pocket for the dimerization arm from the other molecule in the receptor dimer. Additional interactions across the dimer interface from a C-terminal loop on domain II contribute substantially to the stability of the EGFR dimer. The conformation of domain II in this region is stabilized by interactions with domain III that have been demonstrated to be critical for EGFR dimerization and activation (Dawson et al., 2005). The binding of Fab72000 to domain III would disrupt all these interactions.

Fab72000 binding to domain III of EGFR blocks the domain rearrangement and the local conformational changes in domain II. Thus, it is proposed that blocking both of these key elements in formation of the productive EGFR dimer is critical for the effective inhibition of EGFR activation by matuzumab.

### CONCLUSION

EGFR dimerization requires a conformational reorganization of the receptor extracellular region that is promoted by ligand binding to domains I and III (FIG. 3D). Cetuximab acts as a competitive inhibitor, preventing ligand induced dimerization by directly blocking access of ligand to the domain III ligand binding site. By contrast matuzumab does not occlude the ligand binding site on domain III. Rather matuzumab exploits a novel, non-competitive mechanism to inhibit sEGFR dimerization and activation that is entirely dependent on sterically blocking the receptor from adopting the conformation that is required for high affinity ligand binding and dimerization.

This is interesting in the context of a combination of therapeutic antibodies with different inhibition mechanisms to exploit and synergize their respective potentials in cancer treatment.

### EXAMPLES

### Example 1

### Protein Expression and Purification

sEGFR and sEGFRd3 were expressed in baculovirus-infected Sf9 cells, purified as described (Ferguson et al., 2000; Li et al., 2005) and used without modification of their glycosylation state.

Matuzumab (EMD72000) was provided by Merck Serono KGaA (USA/Germany). The Fab fragment (Fab72000) was generated by papain cleavage using the ImmunoPure Fab Preparation Kit (Pierce), and used without further purification. Fab72000/sEGFR complex was generated exactly as described (Li et al., 2005). To generate the complex with sEGFRd3 a Fab was mixed with a 1.2 fold molar excess of sEGFRd3 and excess sEGFRd3 separated from complex by size exclusion chromatography (SEC) using a Bio-Silect SEC250 column (Bio-Rad), equilibrated with 20 mM HEPES, 100 mM NaCl, pH 7.5.

### Example 2

### Crystallization and Data Collection

Proteins were concentrated and buffer exchanged into 10 mM HEPES, 50 mM NaCl, pH 7.5 and crystallized using the hanging drop vapor diffusion method. Large single crystals of Fab72000 were obtained by mixing equal volumes (1 μl) of the Fab (13 mg/ml) with a solution containing 1.8 M ammonium sulfate, 0.1 M MES, pH 6.5 and equilibrating this over a reservoir of this buffer at 20° C. Crystals were flash frozen in reservoir containing 9% sucrose, 2% glucose, 8% glycerol, 8% ethylene glycol. Data were collected at the Cornell High Energy Synchrotron Source (CHESS) beamline F1, using an ADSC Quantum-210 CCD detector. Fab72000/sEGFRd3 was crystallized by mixing equal parts (1 μl) of complex (14 mg/ml) with 1 M NaCl, 16% PEG 3350, 50 mM MES, pH 6.0 and equilibrating over a reservoir of the same buffer at 200° C. Streak seeding was used to produce large single crystals (0.5×0.1×0.15 mm) that were crystostabilized by serial transfer to solutions of reservoir containing increasing concentrations of ethylene glycol (final concentration 15%) and flash frozen in liquid nitrogen. Data were collected at the Swiss Light Source (SLS) beamline X06SA. All data were processed in HKL2000 (Otwinowski and Minor, 1997). Data collection statistics are summarized in Table 1.

### Example 3

### Structure Determination and Refinement

The structures of the Fab72000 and Fab72000/sEGFRd3 were solved by the method of molecular replacement (MR) using the program PHASER (CCP4, 1994). To solve the Fab structure the coordinates for Fab2C4 (PDB id 1 L71) (Vajdos et al., 2002) were selected as the initial search model based on the sequence identity between this Fab and Fab72000. To solve the Fab72000/sEGFRd3 structure the Fab was first located using the refined Fab72000 coordinates as search model. One of the two Fab copies in the asymmetric unit was located. With this solution fixed a second search using the coordinates of domain III of sEGFR (amino acids 310-500 from PDB id 1YY9) located one sEGFRd3 molecule. Subsequently the second Fab72000/sEGFRd3 complex in the asymmetric unit was found. Coordinates were manually rebuilt in COOT (Emsley and Cowtan, 2004) and refined using CNS (Brunger et al., 1998) and Refmac (CCP4, 1998). New maps were calculated following every iteration of refinement, including solvent flattened maps with minimized model bias calculated by the CCP4 program DM.

### Example 4

### Binding Studies

Surface Plasmon Resonance (SPR)/Biacore studies were carried out using a BIAcore 3000 instrument at 25° C. in 10 mM Tris (pH 8.0), 150 mM NaCl, 3 mM EDTA, 0.005% Tween-20. Fab72000 was immobilized on a Biacore CM5 biosensor chip as follows: the CM-dextran matrix was activated with N-ethyl-N′-(dimethylaminopropyl)-carbodiimide hydrochloride (EDC) and N-hydroxysuccinimide. 500 ng Fab fragment were flown over the activated surface at 5 μg/ml in 10 mM sodium acetate, pH 5.0. The remaining reactive sites were blocked with 1M ethanolamine-HCl (pH 8.5). Immobilized Fab fragment contributed a signal of 1436 response units (RU). The surface was regenerated between sEGFR contacts with two 5 μl injections 10 mM glycine (pH 2.5), 1 M NaCl to remove remaining receptor. EGF immobilization and sEGFR binding analysis were performed exactly as described before (Ferguson et al., 2000). Data were analyzed using Prism 4 (GraphPad Software, Inc.).

### Example 5

### Generation of sEGFR Epitope Mutations

Site-directed mutagenesis was used to introduce alanine or deletion mutations in the appropriate DNA in the pFastBac vector. The following mutations were made: K454A, K463A, ΔS460, T459A/S460A, K454A/T459A/S460A, T459A/S460A/K463A. The generation of recombinant baculovirus, overexpression in Sf9 cells and protein purification were exactly as described before for wild-type sEGFR (Ferguson et al., 2000).

Coordinates of the Fab72000 and Fab72000/sEGFRd3 structures have been deposited with the RCSB protein data bank (pdb id codes xxxx and xxxx respectively).

TABLE 1
Table 1. Data collection and refinement statistics

| | |
|---|---|
| Fab72000 | Fab72000/sEGFRd3 |

Data collection statistics^{a}

| | | |
|---|---|---|
| Space group | P2₁2₁2₁ | C2 |
| Unique cell dimensions | a = 56.8 Å, b = 61.4 Å, c = 102.7 Å | a = 141.1 Å, b = 205.0 Å, c = 81.6 |
| | | Å, β = 117.5° |
| X-ray source | CHESS F1 | SLS X06SA |
| Resolution limit | 2.15 Å | 3.2 Å |
| Observed/unique | 107,297/20,191 | 120,206/33,886 |
| Completeness (%) | 99.9 (99.9) | 99.7 (98.7) |
| R_{sym}^{b} | 0.10 (0.42) | 0.12 (0.35) |
| <1/σ> | 20.7 (3.6) | 11.4 (3.4) |

Refinement statistics

| | | |
|---|---|---|
| Resolution limits | 50-2.15 Å | 50-3.2 Å |
| No. of reflections/no. test set | 19,098/1,029 | 32,028/1,709 |
| R factor (R_{free})^{c} | 0.22 (0.26) | 0.24 (0.29) |
| Model | 1 Fab72000 molecule | 2 Fab72000/sEGFRd3 complexes |
| Protein | aa 4-211 (light chain) | aa 310-500 of mature sEGFRd3 with |
| | aa 1-224 (heavy chain) | 13 saccharide units |
| | | aa 1-211 of Fab light chain |
| | | aa 1-222 of Fab heavy chain^{d} |
| Water/Ions | 99 water molecules, 2 sulfates | — |
| Total number of atoms | 3,209 | 8,517 |
| RMSD bond length (Å) | 0.012 | 0.015 |
| RMSD bond angles (°) | 1.35 | 1.6 |

^{a}Numbers in parentheses refer to last resolution shell.
^{b}R_{sym} = Σ|Iₕ − <Iₕ>|/ΣIₕ, where <Iₕ> = average intensity over symmetry equivalent measurements.
^{c}R factor = Σ|Fₒ − F_{c}|/ΣFₒ, where summation is over data used in the refinement; R_{free} includes 5% of the data excluded from the refinement
^{d}Number of missing amino acids at the C-terminal ends of the heavy and light chains differ in the two complexes

TABLE 2
List of residue-receptor/residue-Matuzumab
Fab contacts/interactins:

| source atoms | | | | target atoms | | | | distance | angle |
|---|---|---|---|---|---|---|---|---|---|
| Ser | 433D | O | . . . | His | 93L | CE1 | . . . | 3.35 | |
| | | | . . . | His | 93L | NE2 | . . . | 3.91 | * |
| Asp | 434D | CB | . . . | His | 93L | NE2 | . . . | 3.69 | |
| Asp | 434D | CG | . . . | His | 93L | CE1 | . . . | 3.72 | |
| | | | . . . | His | 93L | NE2 | . . . | 3.02 | |
| Asp | 434D | OD1 | . . . | His | 93L | CD2 | . . . | 3.77 | |
| | | | . . . | His | 93L | CE1 | . . . | 3.18 | |
| | | | . . . | His | 93L | NE2 | . . . | 2.74 | *** |
| Asp | 434D | OD2 | . . . | His | 93L | NE2 | . . . | 3.47 | * |
| Asp | 436D | OD2 | . . . | Tyr | 31L | OH | . . . | 3.91 | * |
| Asn | 449D | N | . . . | Tyr | 103H | OH | . . . | 3.86 | * |
| Asn | 449D | CA | . . . | Tyr | 103H | OH | . . . | 3.49 | |
| Asn | 449D | CG | . . . | Tyr | 103H | OH | . . . | 3.82 | |
| Asn | 449D | ND2 | . . . | Tyr | 103H | CZ | . . . | 3.67 | |
| | | | . . . | Tyr | 103H | OH | . . . | 2.76 | *** |
| Asn | 452D | ND2 | . . . | Ser | 54H | CB | . . . | 3.92 | |
| | | | . . . | Thr | 30H | O | . . . | 3.60 | * |
| Trp | 453D | CD1 | . . . | Tyr | 103H | CE1 | . . . | 3.84 | |
| Lys | 454D | C | . . . | Ser | 54H | OG | . . . | 3.93 | |
| | | | . . . | Asn | 52H | ND2 | . . . | 3.79 | |
| Lys | 454D | O | . . . | Asn | 52H | ND2 | . . . | 2.95 | *** |
| | | | . . . | Asn | 55H | ND2 | . . . | 3.70 | * |
| Lys | 454D | CG | . . . | Ser | 31H | O | . . . | 3.47 | |
| Lys | 454D | CD | . . . | Ser | 31H | O | . . . | 3.53 | |
| | | | . . . | Asp | 100H | OD2 | . . . | 3.76 | |
| Lys | 454D | CE | . . . | Asp | 100H | CG | . . . | 3.49 | |
| | | | . . . | Asp | 100H | OD1 | . . . | 3.80 | |
| | | | . . . | Ser | 31H | O | . . . | 3.20 | |
| | | | . . . | Asp | 100H | OD2 | . . . | 2.64 | |
| | | | . . . | Tyr | 101H | O | . . . | 3.50 | |
| Lys | 454D | NZ | . . . | Asp | 100H | OD2 | . . . | 3.48 | * |
| | | | . . . | Tyr | 101H | C | . . . | 3.68 | |
| | | | . . . | Tyr | 101H | O | . . . | 2.46 | *** |
| Lys | 455D | N | . . . | Ser | 54H | OG | . . . | 3.48 | * |
| Lys | 455D | CA | . . . | Ser | 54H | OG | . . . | 3.24 | |
| | | | . . . | Asn | 55H | ND2 | . . . | 3.92 | |
| Lys | 455D | C | . . . | Asn | 55H | ND2 | . . . | 3.44 | |
| Lys | 455D | O | . . . | Asn | 55H | CB | . . . | 3.55 | |
| | | | . . . | Asn | 55H | CG | . . . | 3.77 | |
| | | | . . . | Asn | 55H | ND2 | . . . | 3.01 | *** |
| Lys | 455D | CB | . . . | Ser | 54H | OG | . . . | 3.64 | |
| Leu | 456D | C | . . . | Arg | 57H | NH1 | . . . | 3.90 | |
| Leu | 456D | O | . . . | Arg | 57H | NH1 | . . . | 3.97 | * |
| Phe | 457D | N | . . . | Arg | 57H | NH1 | . . . | 3.95 | * |
| | | | . . . | Asn | 55H | ND2 | . . . | 3.89 | * |
| Phe | 457D | C | . . . | Arg | 57H | NH1 | . . . | 3.93 | |
| Phe | 457D | O | . . . | Arg | 57H | CD | . . . | 3.59 | |
| | | | . . . | Arg | 57H | NH1 | . . . | 3.52 | * |
| | | | . . . | Asn | 52H | ND2 | . . . | 3.50 | * |
| | | | . . . | Asn | 55H | ND2 | . . . | 3.28 | *** |
| | | | . . . | Trp | 33H | CZ2 | . . . | 3.63 | |
| Gly | 458D | C | . . . | Trp | 33H | CH2 | . . . | 3.49 | |
| | | | . . . | Trp | 33H | CZ2 | . . . | 3.40 | |
| Gly | 458D | O | . . . | Trp | 33H | CH2 | . . . | 3.06 | |
| | | | . . . | Asn | 59H | CG | . . . | 3.97 | |
| | | | . . . | Asn | 59H | ND2 | . . . | 3.16 | *** |
| | | | . . . | Trp | 33H | CZ2 | . . . | 3.45 | |
| Thr | 459D | N | . . . | Trp | 33H | CH2 | . . . | 3.94 | |
| | | | . . . | Trp | 33H | CZ2 | . . . | 3.49 | |
| Thr | 459D | CA | . . . | Trp | 33H | CH2 | . . . | 3.95 | |
| | | | . . . | Trp | 33H | CZ2 | . . . | 3.57 | |
| Thr | 459D | C | . . . | Trp | 33H | NE1 | . . . | 3.51 | |
| | | | . . . | Trp | 33H | CE2 | . . . | 3.46 | |
| | | | . . . | Trp | 33H | CZ2 | . . . | 3.53 | |
| Thr | 459D | O | . . . | Trp | 33H | NE1 | . . . | 2.98 | *** |
| | | | . . . | Trp | 33H | CD1 | . . . | 3.86 | |
| | | | . . . | Trp | 33H | CE2 | . . . | 3.33 | |
| | | | . . . | Trp | 33H | CZ2 | . . . | 3.49 | |
| Thr | 459D | CB | . . . | Trp | 90L | CZ2 | . . . | 3.63 | |
| | | | . . . | His | 93L | ND1 | . . . | 3.81 | |
| Thr | 459D | OG1 | . . . | Trp | 90L | CZ2 | . . . | 3.59 | |
| | | | . . . | His | 93L | ND1 | . . . | 3.33 | * |
| | | | . . . | His | 93L | CE1 | . . . | 3.37 | |
| Thr | 459D | CG2 | . . . | Glu | 50H | OE1 | . . . | 3.19 | |
| | | | . . . | His | 93L | O | . . . | 3.98 | |
| | | | . . . | Trp | 90L | CZ2 | . . . | 3.61 | |
| | | | . . . | His | 93L | CA | . . . | 3.57 | |
| | | | . . . | His | 93L | CB | . . . | 3.80 | |
| | | | . . . | His | 93L | CG | . . . | 3.75 | |
| | | | . . . | His | 93L | ND1 | . . . | 3.12 | |
| | | | . . . | Trp | 90L | NE1 | . . . | 3.76 | |
| Ser | 460D | N | . . . | Trp | 33H | NE1 | . . . | 3.88 | * |
| | | | . . . | Glu | 50H | OE1 | . . . | 3.97 | * |
| | | | . . . | Glu | 50H | OE2 | . . . | 3.35 | * |
| | | | . . . | Trp | 33H | CE2 | . . . | 3.86 | |
| Ser | 460D | CA | . . . | Glu | 50H | OE2 | . . . | 3.96 | |
| Ser | 460D | O | . . . | Tyr | 101H | CD2 | . . . | 3.31 | |
| Ser | 460D | CB | . . . | Glu | 50H | OE2 | . . . | 3.53 | |
| Ser | 460D | OG | . . . | Glu | 50H | CD | . . . | 3.58 | |
| | | | . . . | Glu | 50H | OE2 | . . . | 2.39 | *** |
| | | | . . . | His | 35H | CE1 | . . . | 3.26 | |
| | | | . . . | His | 35H | NE2 | . . . | 3.29 | *** |
| | | | . . . | Arg | 99H | O | . . . | 3.65 | * |
| | | | . . . | Phe | 95L | CE1 | . . . | 3.94 | |
| | | | . . . | Phe | 95L | CZ | . . . | 3.98 | |
| Gly | 461D | N | . . . | Trp | 90L | CZ2 | . . . | 3.97 | |
| Gly | 461D | CA | . . . | Trp | 90L | CZ2 | . . . | 3.80 | |
| | | | . . . | Trp | 90L | CZ3 | . . . | 3.80 | |
| | | | . . . | Trp | 90L | CH2 | . . . | 3.62 | |
| Gly | 461D | O | . . . | Tyr | 31L | CE2 | . . . | 3.62 | |
| | | | . . . | Tyr | 31L | OH | . . . | 3.69 | * |
| Lys | 463D | CD | . . . | Tyr | 101H | CG | . . . | 3.80 | |
| | | | . . . | Tyr | 101H | CD1 | . . . | 3.56 | |
| | | | . . . | Tyr | 101H | CE1 | . . . | 3.73 | |
| Lys | 463D | CE | . . . | Tyr | 31L | CZ | . . . | 3.87 | |
| | | | . . . | Tyr | 31L | OH | . . . | 3.15 | |
| Lys | 463D | NZ | . . . | Tyr | 101H | CD1 | . . . | 3.97 | |
| | | | . . . | Tyr | 101H | CE1 | . . . | 3.34 | |
| | | | . . . | Tyr | 101H | CZ | . . . | 3.74 | |
| | | | . . . | Asp | 49L | OD2 | . . . | 3.42 | * |
| | | | . . . | Tyr | 101H | OH | . . . | 3.89 | * |
| | | | . . . | Tyr | 31L | CZ | . . . | 3.99 | |
| | | | . . . | Tyr | 31L | OH | . . . | 3.75 | * |
| Thr | 464D | O | . . . | Tyr | 103H | O | . . . | 3.74 | * |
| Thr | 464D | OG1 | . . . | Tyr | 103H | O | . . . | 3.79 | * |
| Ile | 466D | CB | . . . | Tyr | 103H | OH | . . . | 3.73 | |

The following literature citations are cited in the description above and are incorporated by reference:
(1) Baselga, J. (2002), Oncologist 7, 2-8.
(2) Bier, H. et al., (2001), Cancer Chemother. Pharmacol. 47, 519-524.
(3) Brünger, A. T. et al., (1998), Acta Crytallogr. D Biol. Crystallogr. 54, 905-921.
(4) Burgess, A. W. et al., Mol. Cell. 12, 541-552.
(5) CCP4 (Collaborative Computational Project, Number 4) (1994). The CCP4 suite: programs for protein crystallography. Acta Crystallogr. D Biol. Crystallogr. 50, 760-763.
(6) Cho, H.-S. et al (2003), Nature 421, 756-60.
(7) Dawson, J. P. et al., (2005). Mol. Cell. Biol. 25, 7734-7742.
(8) Emsley, P. and Cowtan, K. (2004). Acta Crystallogr. D Biol. Crystallogr. 60, 2126-2132.
(9) Ferguson, K. M. et al., (2003), Mol. Cell. 11, 507-517.
(10) Ferguson, K. M. (2004), Biochem. Soc. Trans. 32, 742-745.
(11) Ferguson, K. M., et al., (2000), EMBO J. 19, 4632-4643.
(12) Franklin, M. C. et al., (2004), Cancer Cell 5, 317-328.
(13) Garrett, T. P. J. et al., (2002). Cell 110, 763-773.
(14) Graeven, U. et al., (2006), Br. J. Cancer 94, 1293-1299.
(15) Hubbard, S. R. et al. (2007), Curr. Opin. Cell. Biol. 19, 117-123.
(16) Johns, T. G. et al., (2004), J. Biol. Chem. 279, 30375-30384.
(17) Kettleborough, C. A. et al., (1991), Protein Eng. 4, 773-783.
(18) Lawrence, M. C. and Colman, P. M. (1993), J. Mol. Biol. 234, 946-950.
(19) Kollmannsberger, C., (2006), Ann. Oncol. 17, 1007-1013.
(20) Li, S. et al., (2005), Cancer Cell 7: 301-311.
(21) Mendelsohn, J. and Baselga, J. (2006), Oncol. 33, 369-385.
(22) Murthy, U. et al., (1987), Arch. Biochem. Biophys. 252, 549-560.
(23) Ogiso, H. et al., (2002), Cell 110, 775-787.
(24) Otwinowski, Z. and Minor, W. (1997); In Macromolecular Crystallography, Volume 276, C. W. Carter and R. M. Sweet, eds. (New York: Academic Press), pp 307-326.
(25) Rocha-Lima, C. M. et al., (2007), Cancer Control. 14, 295-304.
(26) Rodeck, U. et al., (1990), J. Cell. Biol. 44, 69-79.
(27) Seiden, M. V. et al., (2007), Gynecol. Oncol. 104, 727-731.
(28) Socinski, M. A. (2007), Clin. Cancer Res. 13, 4597-4601.
(29) Stanfield, R. L. et al., (2006), J. Mol. Biol. 31, 1566-1574.
(30) Steiner, K. et al., (1995), Cell. Mol. Biol. 41, 179-184.
(31) Sundberg, E. J. et al., (2000), Biochemistry 39, 15375-15387.
(32) Vajdos, F. F. et al., (2002), J. Mol. Biol. 320, 415-428.
(33) Sundberg, E. J. et al., (2000), Biochemistry 39, 15375-15387.
(34) Sundberg, E. J. and Mariuzza, R. A. (2002), Adv. Protein Chem. 61, 119-160.
(35) Vanhoefer, U. et al., (2004), J. Clin. Oncol. 22, 175-184.

We claim:

1. A crystal of a receptor-antibody complex comprising a receptor-antibody complex of an epidermal growth factor receptor (EGFR) extracellular domain and matuzumab (EMD 72000) Fab, wherein the crystal belongs to space group C2 and has the following cell coordinates:
a=141 Å(+3 Å), b=205 Å(±3 Å), c=81 Å(±3 Å), and (β=117°(±10).

2. The crystal of claim 1, wherein the crystal has the following cell coordinates:
a=141.1 Å(±0.3 Å), b=205.0 Å(±0.3 Å), c=81.6 Å(±0.3 Å), and (β=117.5°(±0.5°).

3. The crystal of claim 1, wherein the crystal is obtainable by mixing the Fab fragment of matuzumab, generated by papain cleavage of the whole antibody, with an EGFR soluble extracellular domain (sEGFR) in a solution containing 1 M NaCl, 16% PEG 3350, 50 mM MES, pH 6.0-7.0 and equilibrating over a reservoir of the same buffer at about 20° C.

4. The crystal of claim 3, wherein the crystal was obtained by using the soluble extracellular EGFR domain III (sEGFRd3).

5. A method of preparing a crystal of a complex of an EGFR extracellular domain and matuzumab-Fab by preparing a buffered solution of a pH 6.0 to 7.0 containing said EGFR domain and said Fab fragment of matuzumab, and growing the crystal.

6. The method of claim 5, wherein the soluble extracellular EGFR domain III (sEGFRd3) is used.

7. The method of claim 5, wherein the buffered solution contains a precipitation agent.

8. The method of claim 5, wherein the buffered solution contains polyethylene glycol (PEG) as a precipitation agent.

9. A method of identifying and selecting a mimetic candidate of matuzumab, having similar or the same biological activity; said method comprising comparing (a) the binding coordinates of the mimetic compound or a fragment thereof bound to EGFR within the crystalline structure of a mimetic—EGFR complex with (b) the binding coordinates of the Fab fragment of matuzumab bound to EGFR within the crystal of claim 1, wherein said binding coordinates are defined by
(i) positions of amino acid residues of EGFR binding to or interacting with the Fab fragment of matuzumab,
(ii) the distances of specific atoms within the EGFR amino acids of these positions to specific atoms within the mimetic compound that bind or interact with said amino acid residues of EGFR of these positions, and
(iii) for reference, the distances of specific atoms within said EGFR amino acids of these positions to specific atoms within the matuzumab Fab fragment.

10. The method of claim 9, wherein the distances between atoms of the EGFR amino acid residues of said positions and atoms of the mimetic compound binding or interacting with said EGFR amino acid residues of said positions in said crystal complex are less than 4 Å.

11. The method of claim 9, wherein the distances between atoms of the amino EGFR acid residues of said positions and atoms of the mimetic compound binding or interacting with them in said crystal complex are less than 3 Å.

12. The method of claim 9, wherein the crystalline structure of the mimetic—EGFR complex was prepared according to the same or a similar method used for the matuzumab/EGFR crystal complex.

13. The method of claim 9, wherein the amino acid residues of EGFR which are bound to or interact with the mimetic candidate and Fab matuzumab respectively, are selected from the group consisting of:
Ser433, Asp434, Ala448, Asn449, Asn452, Trp453, Lys454, Lys455, Leu456, Phe457, Gly458, Thr459, Ser460, Gly461, Lys463, Thr464 and Ile466.

14. The method of claim 9, wherein the amino acid residues of EGFR which are bound to or interact with the mimetic candidate and Fab matuzumab respectively, are selected from the group consisting of:
Lys454, Lys455, Leu456, Phe457, Gly458, Thr459, Ser460, Gly461, Lys463 and Thr464.

15. The method of claim 9, wherein the amino acid residues of EGFR which are bound to or interact with the mimetic candidate and Fab matuzumab respectively, are selected from the group consisting of:
(a1) Lys454
(a2) Lys454 and Lys455
(a3) Lys454 and Lys455 and Phe457
(a4) Lys454 and Lys455 and Phe457 and Gly458
(a5) Lys454 and Lys455 and Phe457 and Gly458 and Thr459
(a6) Lys454 and Lys455 and Phe457 and Gly458 and Thr459 and Ser460
(a7) Lys454 and Lys455 and Phe457 and Gly458 and Thr459 and Ser460 and Gly461
(a8) Lys454 and Lys455 and Phe457 and Gly458 and Thr459 and Ser460 and Gly461 and Lys463
(a9) Lys454 and Lys455 and Phe457 and Gly458 and Thr459 and Ser460 and Gly461 and Lys463 and Thr464
(a10) Phe457
(a11) Phe457 and Lys454
(a12) Phe457 and Lys454 and Gly458
(a13) Phe457 and Lys454 and Gly458 and Thr459
(a14) Phe457 and Lys454 and Gly458 and Thr459 and Ser460
(a15) Phe457 and Lys454 and Gly458 and Thr459 and Ser460 and Gly461
(a16) Thr459
(a17) Thr459 and Lys454
(a18) Thr459 and Lys454 and Phe457
(a19) Thr459 and Lys454 and Phe457 and Gly458
(a20) Thr459 and Lys454 and Phe457 and Gly458 and Ser460
(a21) Thr459 and Lys454 and Phe457 and Gly458 and Ser460 and Gly461
(a22) Ser460 and Lys454
(a23) Ser460 and Lys454 and Phe457
(a24) Ser460 and Lys454 and Phe457 and Thr459
(a25) Ser460 and Lys454 and Phe457 and Thr459 and Gly461
(a26) Lys454 and Phe457
(a27) Lys454 and Phe457 and Ser460
(a28) Lys454 and Phe457 and Gly461
(a29) Lys454 and Phe457 and Thr459
(a30) Lys454 and Phe457 and Thr459 and Ser460
(a31) Lys454 and Phe457 and Thr459 and Ser460 and Gly461
(a32) Phe457 and Ser460
(a33) Phe457 and Thr459 and Ser460
(a34) Phe457 and Thr459 and Ser460 and Gly461
(a35) Ala448
(a36) Ala448 and Asn449
(a37) Ala448 and Lys454
(a38) Ala448 and Lys454 and Phe457
(a39) Ala448 and Lys454 and Phe457 and Thr459
(a40) Ala448 and Lys454 and Phe457 and Ser460
(a41) Ala448 and Lys454 and Phe457 and Gly461
(a42) Ala448 and Lys454 and Phe457 and Thr459 and Ser460
(a43) Ala448 and Lys454 and Phe457 and Thr459 and Ser460 and Gly461
(a44) Asn449
(a45) Asn449 and Lys454
(a46) Asn449 and Lys454 and Phe457
(a47) Asn449 and Lys454 and Phe457 and Thr459
(a48) Asn449 and Lys454 and Phe457 and Thr459 and Ser460
(a49) Asn449 and Lys454 and Phe457 and Thr459 and Ser460 and Gly461
(a50) Asn449 and Lys454 and Phe457 and Ser460
(a51) any of the elections (a1)-(a50) with Ser433 and/or Asp433.

16. The method of claim 9 wherein the mimetic compound is a peptide, a polypeptide or a protein.

17. The method of claim 9, wherein the mimetic compound is an antibody or a CDR-containing fragment thereof.

18. The method of claim 9, wherein the mimetic compound binds to EGFR with similar or improved affinity as the Fab fragment of matuzumab.

19. The method of claim 9, wherein the mimetic compound has at least one the following properties:
(i) inhibits tyrosine kinase activity of the EGFR,
(ii) inhibits dimerization of the EGFR,
(iii) blocks binding of EGF to the EGFR.

20. The method of claim 19, wherein the mimetic compound is an antibody or a CDR-containing fragment thereof.

21. The method of claim 9, the method further comprising
(i) modifying the mimetic candidate before forming the crystalline complex with EGFR,
(ii) determining said binding coordinates and
(iii) selecting the mimetic candidate that has improved binding properties to one or more EGFR amino acid residues of said positions in said crystalline structure.

22. The method of claim 21, wherein the mimetic candidate has maintained or obtained the biological activity of an EGFR antagonist as specified in claim 20.

23. The method of claim 22, wherein the mimetic candidate is an antibody or fragment thereof, and the modification is carried out by substitution of at least one amino acid within at least one CDR region of said antibody or fragment thereof.

24. A compound, which is a matuzumab (EMD 72000) mimetic, and
(i) inhibits tyrosine kinase activity of the EGF receptor,
(ii) inhibits dimerization of EGF receptor,
(iii) blocks binding of EGF to EGF receptor, and
(iv) binds or interacts with at least three amino acid residues between position 448 and 464 of EGFR.

25. The compound of claim 24, which binds or interacts at least with three amino acid residues between position 454 and 459 of EGFR.

26. The compound of claim 24, which binds or interacts at least with the following residues of EGFR:
Lys454, Lys455, Leu456, Phe457, Gly458, Thr459, Ser460, Gly461, Lys463 and Thr464.

27. The compound of claim 24, which binds or interacts at least with the following residues of EGFR:
Lys454, Lys455, Leu456, Phe457, Gly458 and Thr459.

28. A composition of compounds comprising at least the compound of claim 27 and a second compound that
(i) inhibits tyrosine kinase activity of the EGF receptor,
(ii) inhibits dimerization of EGF receptor,
(iii) blocks binding of EGF to EGF receptor, and
(iv) binds or interacts with the following amino acid residues of EGFR or a subset of at least four amino acids:
Gln 384, Gln 408, Ser 418, Ser 440, Lys 465, Ser 468, and Asn 469.

29. The composition of claim 28, wherein said second compound is cetuximab or a cetuximab mimetic compound binding or interacting at least with the same EGFR amino acid residues.

30. A method of designing an anti-EGFR antibody that
inhibits tyrosine kinase activity of the EGF receptor,
inhibits dimerization of EGF receptor,
blocks binding of EGF to EGF receptor, and
derives from matuzumab (EMD 72000),
the method comprising:
(i) substituting at least a single amino acid in the CDR regions of the heavy and/or light chain of matuzumab or in the framework regions (FR) adjacent to the CDRs with another amino acid;
(ii) preparing a crystalline complex formed by the Fab fragment of said modified matuzumab and the soluble extracellular domain III of EGFR (sEGFRd3);
(iii) comparing the atomic coordinates of the so-formed crystalline complex with respect to specific amino acid residue positions within the EGFR domain with the corresponding coordinates of a reference crystal complex formed by the non-modified matuzumab with sEGFRd3;
(iv) selecting the modified matuzumab with atomic coordinates that provide closer interaction with respect to said specific amino acid residue positions in the EGFR domain;
(v) assaying its binding affinity and biological activity by means of standard methods; and
(vi) optionally repeating steps (i) to (v).

31. The method of claim 30, wherein the respective crystalline complex was obtained by mixing the Fab fragment of modified or non-modified matuzumab, generated by papain cleavage of the respective whole antibody, with an EGFR soluble extracellular domain (sEGFR) in a buffered solution containing 1 M NaCl, 16% PEG 3350, 50 mM MES, pH 6.0-7.0 and equilibrating over a reservoir of the same buffer at about 20° C., wherein the buffered solution contains polyethylene glycol (PEG) as a precipitation agent.

32. The method of claim 30, wherein at least four of said specific amino acid residue positions within the EGFR domain are selected from the group consisting of:
Ser433, Asp434, Ala448, Asn449, Asn452, Trp453, Lys454, Lys455, Leu456, Phe457, Gly458, Thr459, Ser460, Gly461, Lys463, Thr464 and Ile466.

33. The method of claim 30, wherein at least four of said specific amino acid residue positions within the EGFR domain are selected from the group consisting of:
Lys454, Lys455, Leu456, Phe457, Gly458, Thr459, Ser460, Gly461, Lys463 and Thr464.

34. The method of claim 30, wherein said specific amino acid residue positions within the EGFR domain are at least selected from the group consisting of:
(a1) Lys454
(a2) Lys454 and Lys455
(a3) Lys454 and Lys455 and Phe457
(a4) Lys454 and Lys455 and Phe457 and Gly458
(a5) Lys454 and Lys455 and Phe457 and Gly458 and Thr459
(a6) Lys454 and Lys455 and Phe457 and Gly458 and Thr459 and Ser460
(a7) Lys454 and Lys455 and Phe457 and Gly458 and Thr459 and Ser460 and Gly461
(a8) Lys454 and Lys455 and Phe457 and Gly458 and Thr459 and Ser460 and Gly461 and Lys463
(a9) Lys454 and Lys455 and Phe457 and Gly458 and Thr459 and Ser460 and Gly461 and Lys463 and Thr464
(a10) Phe457
(a11) Phe457 and Lys454
(a12) Phe457 and Lys454 and Gly458
(a13) Phe457 and Lys454 and Gly458 and Thr459
(a14) Phe457 and Lys454 and Gly458 and Thr459 and Ser460
(a15) Phe457 and Lys454 and Gly458 and Thr459 and Ser460 and Gly461
(a16) Thr459
(a17) Thr459 and Lys454
(a18) Thr459 and Lys454 and Phe457
(a19) Thr459 and Lys454 and Phe457 and Gly458
(a20) Thr459 and Lys454 and Phe457 and Gly458 and Ser460
(a21) Thr459 and Lys454 and Phe457 and Gly458 and Ser460 and Gly461
(a22) Ser460 and Lys454
(a23) Ser460 and Lys454 and Phe457
(a24) Ser460 and Lys454 and Phe457 and Thr459
(a25) Ser460 and Lys454 and Phe457 and Thr459 and Gly461
(a26) Lys454 and Phe457
(a27) Lys454 and Phe457 and Ser460
(a28) Lys454 and Phe457 and Gly461
(a29) Lys454 and Phe457 and Thr459
(a30) Lys454 and Phe457 and Thr459 and Ser460
(a31) Lys454 and Phe457 and Thr459 and Ser460 and Gly461
(a32) Phe457 and Ser460
(a33) Phe457 and Thr459 and Ser460
(a34) Phe457 and Thr459 and Ser460 and Gly461
(a35) Ala448
(a36) Ala448 and Asn449
(a37) Ala448 and Lys454
(a38) Ala448 and Lys454 and Phe457
(a39) Ala448 and Lys454 and Phe457 and Thr459
(a40) Ala448 and Lys454 and Phe457 and Ser460
(a41) Ala448 and Lys454 and Phe457 and Gly461
(a42) Ala448 and Lys454 and Phe457 and Thr459 and Ser460
(a43) Ala448 and Lys454 and Phe457 and Thr459 and Ser460 and Gly461
(a44) Asn449
(a45) Asn449 and Lys454
(a46) Asn449 and Lys454 and Phe457
(a47) Asn449 and Lys454 and Phe457 and Thr459
(a48) Asn449 and Lys454 and Phe457 and Thr459 and Ser460
(a49) Asn449 and Lys454 and Phe457 and Thr459 and Ser460 and Gly461
(a50) Asn449 and Lys454 and Phe457 and Ser460
(a51) any of the elections (a1)-(a50) with Ser433 and/or Asp433.

35. The method of claim 30, wherein the atomic coordinates of the crystal complex formed by modified Fab matuzumab and sEGFRd3 result in atomic distances between the amino acid residues participated in interaction or binding less than the respective distances provided by the atomic coordinates of the reference crystal complex formed by non-modified or less modified matuzumab.

36. The method of claim 35, wherein the respective distances provided by the atomic coordinates are less than 4 Å.

37. The method of claim 35, wherein the respective distances provided by the atomic coordinates are less than 3 Å.

38. The method of claim 35, wherein the respective distances provided by the atomic coordinates are less than 2 Å.

39. The method of claim 30, wherein the substitution is carried out in the CDRs or FRs of the heavy chain of matuzumab.

40. The method of claim 30, wherein substitution is carried out in the CDR3 of the heavy or light chain of matuzumab.

41. An anti-EGFR antibody obtained by the method of claim 30.

42. A method of treating EGFR-related diseases or disorders by inhibiting EGFR comprising administering to a patient the compound of claim 24.

43. A method of claim 42, wherein the EGFR-related disease or disorder is cancer.

44. A method of designing and manufacturing an anti-EGFR antibody derived from matuzumab (EMD 72000), comprising using at least three amino acid residues Ser433, Asp434, Ala448, Asn449, Asn452, Trp453, Lys454, Lys455, Leu456, Phe457, Gly458, Thr459, Ser460, Gly461, Lys463, Thr464 and Ile466 within an EGFR antibody crystal complex to design and manufacture the anti-EGFR antibody.

45. The method of claim 44, comprising using the amino acid residues Lys454, Lys455, Leu456, Phe457, Gly458, Thr459, Ser460 and Gly461 within an EGFR antibody crystal complex to design and manufacture the anti-EGFR antibody.


SEQUENCE LISTING (2 sequences)

SEQ ID NO: 1 - protein, 213 aa, Artificial
  Humanized monoclonal antibody
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 451 aa, Artificial
  Humanized monoclonal antibody
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK